(19) United States
(12) Patent Application Publication
(10) Pub. No.: US 2023/0021801 A1
(43) Pub. Date: Jan. 26, 2023

(54) Method for Selective Modulation

(71) Applicant: Galvani Bioelectronics Limited, Brentford, Middlesex (GB)

(72) Inventors: Arun Sridhar, Brentford (GB); Philip Milliken, Brentford (GB)

(21) Appl. No.: 17/782,539

(22) PCT Filed: Dec. 9, 2020
(86) PCT No.: PCT/GB2020/053146

§ 371 Date: Jun. 3, 2022

(30) Foreign Application Priority Data
Dec. 11, 2019 (GB) 1918168.4

(51) Int. Cl. A61N 1/36

(52) U.S. Cl. CPC A61N 1/36007; A61N 1/3606

(57) ABSTRACT

Provided herein is a solution to the problem of stimulation of a target pudendal nerve such that the stimulation applied by the electrode at that position selectively modulates the external urtheral sphincter (EUS), or selectively modulates the external anal sphincter (EAS).

### METHOD FOR SELECTIVE MODULATION

Bladder function involves control of the bladder filling and voiding phases mediated by continence and micturition reflexes accomplished through coordinated sympathetic, parasympathetic and somatic neural activity [Beckel and Holstege Neurophysiology of the Lower Urinary Tract, in Urinary Tract (2011) Springer Berlin Heldelberg, 149-169]. In bladder dysfunction (for example, over-active bladder (OAB), underactive bladder (UAB) or urinary retention), one or more of these functions is disrupted, leading to symptoms including urinary urgency, frequency, urgency incontinence, nocturia, sensation of incomplete emptying, straining to void, and recurrent infections. These symptoms often fail to improve following pharmacological treatment alone (Izett et al. Minerva Ginecol. 2017 June; 69(3):269-285; McDonnell B and Birder, L A, Version 1. F1000Res. 2017; 6: 2148).

Pudendal nerve stimulation is a promising therapeutic option for treatment of bladder dysfunction symptoms and for treatment of faecal incontinence.

To date, positioning of an electrode for pudendal nerve stimulation has been performed by visual inspection of the pelvic anatomy and neural branch points. However, when using this method alone the complexities of the pudendal nerve branching and its variation between individuals can mean that the stimulation applied by the electrode elicits multiple physiological responses, some of which may be unwanted.

### SUMMARY OF INVENTION

Provided herein is a method which addresses the problem of stimulation of a target pudendal nerve such that the stimulation applied by the electrode at that position selectively modulates the external urtheral sphincter (EUS), or selectively modulates the external anal sphincter (EAS).

In particular, the inventors have identified that by measuring a first physiological response that should be triggered by stimulation when the electrode is positioned for selective modulation of the EUS or, alternatively, the EAS, and measuring a second physiological response that should not be triggered by stimulation when the electrode is positioned for selective modulation of the EUS or EAS, a method of selective modulation is provided such that stimulation of a pudendal nerve by a signal applied by the electrode selectively modulates the EUS or selectively modulates the EAS. Put another way, the methods provided herein provide for selective modulation of the EUS or EAS.

In a first aspect is provided a method of selectively modulating the external urethral sphincter (EUS) or external anal sphincter (EAS) of a subject, the method comprising:
(a) positioning an electrode for stimulating a first target pudendal nerve in a subject, wherein the electrode is configured to apply a stimulatory electrical signal to stimulate a target pudendal nerve;
(b) causing the electrode to apply a first stimulatory electrical signal, such that the electrode stimulates the first target pudendal nerve;
(c) detecting the magnitude of a first physiological response and the magnitude of a second physiological response in response to the first stimulatory electrical signal applied by an electrode;
(d) detecting the presence or absence of selective modulation of the EUS or EAS, wherein:
   i. selective modulation of the EUS or EAS is absent when the magnitude of the first physiological response is detected to be not beyond a first predetermined threshold and/or the magnitude of the second physiological response is detected to be beyond a second predetermined threshold;
   ii. selective modulation of the EUS or EAS is present when the first physiological response is detected to be beyond the first predetermined threshold and the second physiological response is detected to be not beyond the second predetermined threshold;
(e) if selective modulation of the EUS or EAS is detected to be absent:
   iii. re-positioning the electrode to stimulate a second target pudendal nerve;
   and/or
   iv. causing the electrode to apply a second stimulatory signal, such that the electrode stimulates the target pudendal nerve, where the second stimulatory signal differs from the first stimulatory signal by at least one parameter;
(f) repeating steps (a)-(e) until the selective modulation of the EUS or EAS is detected as present.

Provided herein is thus a method whereby the position of an electrode on a pudendal nerve branch, the stimulatory signal applied by the electrode, or both, are modified iteratively such that by the end of the method the signal applied by the electrode in that position results in selective modulation of the intended target—either the EUS or the EAS.

The problem of effectively co-ordinating the position of, and signal applied by, an electrode to selectively modulate the external urtheral sphincter (EUS) is particularly relevant to the treatment of bladder dysfunction, for example underactive bladder (UAB) and/or urinary retention, where selective engagement of the EUS can promote treatment efficacy (e.g. voiding efficiency) and patient comfort.

Therefore in a further aspect is provided a method of treating a subject with bladder dysfunction by selective modulation of the EUS, the method comprising:
(a) identifying a first target pudendal nerve in a subject who has been diagnosed with bladder dysfunction:
(b) positioning an electrode for stimulating the first target pudendal nerve in the subject, wherein the electrode is configured to apply a stimulatory electrical signal to stimulate a target pudendal nerve;
(c) causing the electrode to apply a first stimulatory electrical signal, such that the electrode stimulates the first target pudendal nerve;
(d) detecting the magnitude of a first physiological response and the magnitude of a second physiological response in response to the first stimulatory electrical signal applied by an electrode;
(e) detecting the presence or absence of selective modulation of the EUS, wherein:
   i. selective modulation of the EUS is absent when the magnitude of the first physiological response is detected to be not beyond a first predetermined threshold and/or the magnitude of the second physiological response is detected to be beyond a second predetermined threshold;
   ii. selective modulation of the EUS is present when the first physiological response is detected to be beyond the first predetermined threshold and the second physiological response is detected to be not beyond the second predetermined threshold;
(f) if selective modulation of the EUS is detected to be absent:
   iii. re-positioning the electrode to stimulate a second target pudendal nerve;
   and/or
   iv. causing the electrode to apply a second stimulatory signal, such that the electrode stimulates the target pudendal nerve, where the second stimulatory signal differs from the first stimulatory signal by at least one parameter;
(g) repeating steps (a)-(f) until the selective modulation of the EUS is detected as present;
(h) treating the bladder dysfunction in the subject by causing an electrode to apply a stimulatory signal, where the electrode is at the position in step (g) that resulted in selective modulation of the EUS.

The problem of effectively co-ordinating the position of and/or the signal applied by an electrode to selectively modulate the external anal sphincter (EAS) is particularly relevant to the treatment of faecal incontinence, where selective engagement of the EAS can promote continence and patient comfort. Treatment of faecal incontinence may, for example, reduce diarrhea and/or uncontrolled leakage of stool and/or the number of bowel accidents and/or the number of unexpected leaks (for example, liquid stool, solid stool and/or mucus).

Therefore in a further aspect is provided a method of treating a subject with faecal incontinence by selective modulation of the EAS, the method comprising:
(a) identifying a first target pudendal nerve in a subject who has been diagnosed with faecal incontinence:
(b) positioning an electrode for stimulating the first target pudendal nerve in the subject, wherein the electrode is configured to apply a stimulatory electrical signal to stimulate a target pudendal nerve;
(c) causing the electrode to apply a first stimulatory electrical signal, such that the electrode stimulates the first target pudendal nerve;
(d) detecting the magnitude of a first physiological response and the magnitude of a second physiological response in response to the first stimulatory electrical signal applied by an electrode;
(e) detecting the presence or absence of selective modulation of the EAS, wherein:
   i. selective modulation of the EAS is absent when the magnitude of the first physiological response is detected to be not beyond a first predetermined threshold and/or the magnitude of the second physiological response is detected to be beyond a second predetermined threshold;
   ii. selective modulation of the EAS is present when the first physiological response is detected to be beyond the first predetermined threshold and the second physiological response is detected to be not beyond the second predetermined threshold;
(f) if selective modulation of the EAS is detected to be absent:
   iii. re-positioning the electrode to stimulate a second target pudendal nerve;
   and/or
   iv. causing the electrode to apply a second stimulatory signal, such that the electrode stimulates the target pudendal nerve, where the second stimulatory signal differs from the first stimulatory signal by at least one parameter;
(g) repeating steps (a)-(f) until the selective modulation of the EAS is detected as present;
(h) treating the faecal incontinence in the subject by causing an electrode to apply a stimulatory signal, where the electrode is at the position in step (g) that resulted in selective modulation of the EAS.

Each aspect or embodiment provided herein may be combined with any other aspect(s) or embodiment(s) unless indicated to the contrary. In particular, any feature indicated as being preferred or advantageous may be combined with any other feature or features, most-preferably those indicated as being preferred or advantageous.

### FIGURES

FIG. 1 EMG trace demonstrating effects of point stimulation at differing positions on pudendal branch 1. Non-selective effects were recorded on the EMG signals correlating with visual observations on the EUS, EAS, and pelvic regions, as demonstrated on the left side of the chart. Moving the stimulation location altered the profile, only affecting the EUS and pelvic area, as shown on the right-side trace in the chart. No stimulation (stimulation may be abbreviated as “stim”) waveforms were recorded as this assessment employed a point stimulator.

FIG. 2 Increasing stimulation amplitude with the Adtech interface on pudendal branch 3 (stimulation waveform panel), did not affect EAS or pelvic EMG, but produced amplitude dependent engagement of the EUS EMG (EUS EMG panel). An EUS contraction and EMG firing response occurred when stimulation stopped, further confirming stimulation related EMG activation.

FIG. 3 Expanded section of FIG. 2 demonstrating the bursting stimulation pattern (second trace—“Stim Waveform”) and associated EUS EMG engagement and sinus waveform of activation (third trace—“EUS EMG”).

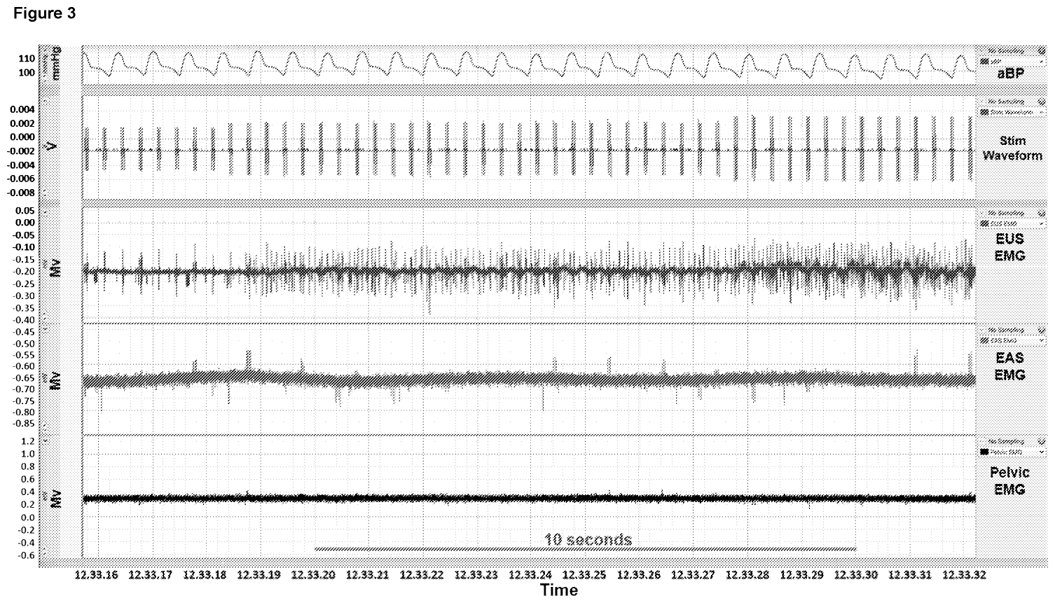

### DETAILED DESCRIPTION

In a first aspect is provided a method of selectively modulating the external urethral sphincter (EUS) or external anal sphincter (EAS) of a subject, the method comprising:
(a) positioning an electrode for stimulating a first target pudendal nerve in a subject, wherein the electrode is configured to apply a stimulatory electrical signal to stimulate a target pudendal nerve;
(b) causing the electrode to apply a first stimulatory electrical signal, such that the electrode stimulates the first target pudendal nerve;
(c) detecting the magnitude of a first physiological response and the magnitude of a second physiological response in response to the first stimulatory electrical signal applied by an electrode;
(d) detecting the presence or absence of selective modulation of the EUS or EAS, wherein:
   i. selective modulation of the EUS or EAS is absent when the magnitude of the first physiological response is detected to be not beyond a first predetermined threshold and/or the magnitude of the second physiological response is detected to be beyond a second predetermined threshold;
   ii. selective modulation of the EUS or EAS is present when the first physiological response is detected to be beyond the first predetermined threshold and the second physiological response is detected to be not beyond the second predetermined threshold;
(e) if selective modulation of the EUS or EAS is detected to be absent:
   iii. re-positioning the electrode to stimulate a second target pudendal nerve;
   and/or
   iv. causing the electrode to apply a second stimulatory signal, such that the electrode stimulates the target pudendal nerve, where the second stimulatory signal differs from the first stimulatory signal by at least one parameter;
(f) repeating steps (a)-(e) until the selective modulation of the EUS or EAS is detected as present.

Provided herein is thus a method whereby the position of an electrode on a pudendal nerve branch, the stimulatory signal applied by the electrode, or both, are modified iteratively such that by the end of the method the signal applied by the electrode in that position results in selective modulation of the intended target—either the EUS or the EAS.

The problem of effectively co-ordinating the position of, and signal applied by, an electrode to selectively modulate the external urtheral sphincter (EUS) is particularly relevant to the treatment of bladder dysfunction, for example underactive bladder (UAB) and/or urinary retention, where selective engagement of the EUS can promote treatment efficacy (e.g. voiding efficiency) and patient comfort.

Therefore in a further aspect is provided a method of treating a subject with bladder dysfunction by selective modulation of the EUS, the method comprising:
(a) identifying a first target pudendal nerve in a subject who has been diagnosed with bladder dysfunction:
(b) positioning an electrode for stimulating the first target pudendal nerve in the subject, wherein the electrode is configured to apply a stimulatory electrical signal to stimulate a target pudendal nerve;
(c) causing the electrode to apply a first stimulatory electrical signal, such that the electrode stimulates the first target pudendal nerve;
(d) detecting the magnitude of a first physiological response and the magnitude of a second physiological response in response to the first stimulatory electrical signal applied by an electrode;
(e) detecting the presence or absence of selective modulation of the EUS, wherein:
   i. selective modulation of the EUS is absent when the magnitude of the first physiological response is detected to be not beyond a first predetermined threshold and/or the magnitude of the second physiological response is detected to be beyond a second predetermined threshold;
   ii. selective modulation of the EUS is present when the first physiological response is detected to be beyond the first predetermined threshold and the second physiological response is detected to be not beyond the second predetermined threshold;
(f) if selective modulation of the EUS is detected to be absent:
   iii. re-positioning the electrode to stimulate a second target pudendal nerve;
   and/or
   iv. causing the electrode to apply a second stimulatory signal, such that the electrode stimulates the target pudendal nerve, where the second stimulatory signal differs from the first stimulatory signal by at least one parameter;
(g) repeating steps (a)-(f) until the selective modulation of the EUS is detected as present;
(h) treating the bladder dysfunction in the subject by causing an electrode to apply a stimulatory signal, where the electrode is at the position in step (g) that resulted in selective modulation of the EUS.

In certain embodiments, the method of treating bladder dysfunction is a method of treating underactive bladder (UAB). In certain embodiments the method of treating bladder dysfunction is a method of treating urinary retention (UR). For example, a method of treating UAB or urinary retention will increase voiding efficiency and/or voiding volume. In another example, a method of treating UAB and urinary retention will reduce the post void residual volume. In a further example, a method of treating UAB and urinary retention may decrease duration of voiding.

The problem of effectively co-ordinating the position of and/or the signal applied by an electrode to selectively modulate the external anal sphincter (EAS) is particularly relevant to the treatment of faecal incontinence, where selective engagement of the EAS can promote continence and patient comfort. Treatment of faecal incontinence may, for example, reduce diarrhea and/or uncontrolled leakage of stool and/or the number of bowel accidents and/or the number of unexpected leaks (for example, liquid stool, solid stool and/or mucus).

Therefore in a further aspect is provided a method of treating a subject with faecal incontinence by selective modulation of the EAS, the method comprising:
(a) identifying a first target pudendal nerve in a subject who has been diagnosed with faecal incontinence:
(b) positioning an electrode for stimulating the first target pudendal nerve in the subject, wherein the electrode is configured to apply a stimulatory electrical signal to stimulate a target pudendal nerve;
(c) causing the electrode to apply a first stimulatory electrical signal, such that the electrode stimulates the first target pudendal nerve;
(d) detecting the magnitude of a first physiological response and the magnitude of a second physiological response in response to the first stimulatory electrical signal applied by an electrode;
(e) detecting the presence or absence of selective modulation of the EAS, wherein:
   i. selective modulation of the EAS is absent when the magnitude of the first physiological response is detected to be not beyond a first predetermined threshold and/or the magnitude of the second physiological response is detected to be beyond a second predetermined threshold;
   ii. selective modulation of the EAS is present when the first physiological response is detected to be beyond the first predetermined threshold and the second physiological response is detected to be not beyond the second predetermined threshold;
(f) if selective modulation of the EAS is detected to be absent:
   iii. re-positioning the electrode to stimulate a second target pudendal nerve;
   and/or
   iv. causing the electrode to apply a second stimulatory signal, such that the electrode stimulates the target pudendal nerve, where the second stimulatory signal differs from the first stimulatory signal by at least one parameter;
(g) repeating steps (a)-(f) until the selective modulation of the EAS is detected as present;
(h) treating the faecal incontinence in the subject by causing an electrode to apply a stimulatory signal, where the electrode is at the position in step (g) that resulted in selective modulation of the EAS.

The principle of the method can be enacted by detecting the level or magnitude of the first and second physiological response in response to stimulation. By detecting whether stimulation results in a change in the level of activity for the first and second physiological response that is greater or lesser than a predetermined threshold, electrode positioning and/or the parameters of the stimulatory signal can be adjusted until the desired selective modulation is achieved. For example, for a particular physiological response the predetermined threshold may be a natural or baseline level of ongoing activity.

In embodiments of all methods provided herein, the first predetermined threshold may be set as the baseline or natural level of activity detectable for the first physiological response, which may be none or zero—i.e. no detectable activity. Alternatively the first predetermined threshold may be a minimum level or change of activity required to give a reliable indication that application of the electrical signal modulates the desired target—i.e. the EUS or the EAS.

For example, where it is desired to selectively modulate the EUS, the first predetermined threshold may be the minimum increase in external urethral sphincter (EUS) activity (e.g. EMG activity) required to give a reliable indication that EUS activity has been modulated in response to the target pudendal nerve (e.g. a perineal nerve branch/branches of the pudendal nerve) being stimulated by the application of the electrical signal. By way of further example, the first predetermined threshold may be the minimum decrease in bladder pressure to give a reliable indication that bladder voiding has been stimulated in reaction to the application of the electrical signal.

By way of further example, where it is desired to selectively modulate the EAS, the first predetermined threshold may be the minimum increase in external anal sphincter (EAS) activity (e.g. EMG activity) required to give a reliable indication that EAS activity has been modulated in response to the target pudendal nerve being stimulated by the application of the electrical signal.

In further embodiments, the second predetermined threshold may be set as the baseline or natural level of activity detectable for the second physiological response, which may be none or zero—i.e. no detectable activity. Alternatively the second predetermined threshold may be set as the maximum level or change of activity that is acceptable for nonspecific stimulation, for example as the threshold at which adverse effects caused by stimulation are typically experienced by a subject. For example, the second predetermined threshold may be set as a given multiple of the natural or baseline level of activity for the second physiological response, e.g. 1.1, 1.2, 1.3, 1.4, 1.5, 1.6, 1.7, 1.8, 1.9 or 2 times the baseline or natural level of activity where nonspecific stimulation is indicated by an increase in the second physiological response. By way of further example, the second predetermined threshold may be set as a given multiple of the natural or baseline level of activity for the second physiological response, e.g. 0.1, 0.2, 0.3, 0.4, 0.5, 0.6, 0.7, 0.8, 0.9 or 1 times the baseline or natural level of activity where nonspecific stimulation is indicated by a decrease in the second physiological response

The pudendal nerve is a complex and branching nerve, and includes the sacral root (S1, S2, S3 or S4), the compound pudendal nerve, and the distal branches such as the dorsal genital nerve (DGN), the perineal nerve and the inferior rectal nerve. The present methods are effectively used for any of these components of the pudendal nerve as the “target pudendal nerve”.

Stimulation of the distal branches of the pudendal nerve is a promising method of treating disorders such as bladder dysfunction (as described in WO2017066572, which is incorporated herein by reference) and faecal incontinence.

Positioning of the electrode and/or selecting the parameters applied by the electrode such that stimulation of distal branches of the pudendal nerve (e.g. a motor pudendal nerve such as the perineal nerve) selectively stimulates the EUS is desirable in order to achieve the intended therapeutic effect while limiting nonspecific stimulation that may cause discomfort or undesirable responses for the subject (for example unwanted stimulation of the pelvic floor muscle).

Selective modulation of the EUS promotes healthy bladder function, for example by promoting bladder contractions and improving voiding efficiency, dysfunction of which is characteristic of underactive bladder (UAB) (see WO2017066572, the contents and embodiments of which are incorporated by reference).

Accordingly, in certain embodiments, the provided methods are methods of treating underactive bladder (UAB) or urinary retention (UR). For example, a method of treating UAB or urinary retention will increase voiding efficiency and/or voiding volume. In another example, a method of treating UAB and urinary retention will reduce the post void residual volume. In a further example, a method of treating UAB and urinary retention may decrease duration of voiding.

Similarly, positioning of the electrode and/or selecting the parameters applied by the electrode such that stimulation of distal branches of the pudendal nerve (e.g. the inferior rectal nerve) selectively stimulates the EAS in order to achieve the intended therapeutic effect (e.g. promoting faecal continence) while limiting nonspecific stimulation that may cause discomfort or undesirable responses for the subject.

Accordingly, in certain embodiments, the provided methods are methods of treating faecal incontinence. Treatment of faecal incontinence may, for example, reduce diarrhea and/or uncontrolled leakage of stool and/or the number of bowel accidents and/or the number of unexpected leaks (for example, liquid stool, solid stool and/or mucus).

Positioning of electrodes for stimulation of distal branches of the pudendal nerve in order to achieve the desired selective modulation is particularly challenging. The skilled person can select the branch that is considered most-likely to be the target pudendal nerve based on visualisation of anatomical landmarks (e.g via direct visualisation, for example during surgery, or via ultrasound). However, the complex branching of this nerve means the skilled person needs to determine that stimulation of the selected branch (the “first target pudendal nerve”) will achieve the desired selective modulation—i.e. of the EUS or EAS. If not, in accordance with the methods provided herein, the electrode can repositioned to stimulate a new branch or a new site on the initial branch—the “second target pudendal nerve” in accordance with the methods provided herein.

Repositioning the electrode in accordance with the methods of the invention is taken to mean changing the site at which the stimulatory signal is applied. In certain embodiments, re-positioning the electrode is done by physically moving the electrode, for example if it is a point electrode. In certain embodiments, the repositioning of the electrode is done by changing the stimulation field of the electrode used to apply the signal. For instance, as demonstrated in the Examples, an electrode comprising multiple contacts can be repositioned in accordance with the methods by changing the multiple contacts used to apply the signal.

In certain embodiments, once the positioning of the electrode selectively modulates the EUS or EAS in accordance with the methods, the electrode used in the method can be fixed and/or implanted at that position in order to apply a stimulatory signal to the pudendal nerve at that position.

In certain alternative embodiments, the positioning for an electrode can be determined according to the methods of the invention. Subsequently, the electrode can be removed and replaced with a second electrode which is fixed and/or implanted at the position for selective modulation of the EUS or EAS. For example, the first electrode may be an electrode better suited to the method of determining the positioning and/or signal parameters required for selective modulation of the EUS, whereas the second electrode may be better suited to applying a stimulatory signal to stimulate the nerve to treat a bladder dysfunction. By way of further example, the first electrode may be an electrode better suited to the method of determining the positioning and/or signal parameters required for selective modulation of the EAS, whereas the second electrode may be better suited to applying a stimulatory signal to stimulate the nerve to treat faecal incontinence. For example, in such embodiments the first electrode may be suitable for point monophasic stimulation, and the second electrode may be suitable for biphasic stimulation.

As described above, the present methods provide an effective means for positioning of the electrode for selective modulation of the EUS or EAS that is more accurate than conventional positioning by anatomical landmarks only.

Preferably the first and/or second target pudendal nerve is located in the pudendal canal or is distally-located relative to the pudendal canal.

In certain preferred embodiments, the target pudendal nerve is a pudendal motor nerve, for example an inferior rectal nerve or a perineal nerve. In certain preferred embodiments, the target pudendal nerve is a perineal nerve. In certain preferred embodiments, the target pudendal nerve is an inferior rectal nerve.

The inventors have identified that stimulation of a target pudendal nerve which innervates the EUS, the pelvic floor, and/or the anal sphincter induces a different response depending on the fibers of the nerve innervating the pelvic floor, one sphincter, the other, both sphincters, or the EUS and the pelvic floor.

The inventors have identified a method enabling selective modulation of the EUS such that the anal sphincter and/or pelvic floor is/are not modulated. The different responses induced by such selective modulation allow a high level of resolution between those pudendal branches that innervate the EUS, and those pudendal branches that innervate the anal sphincter or pelvic floor, or those that innervate both the EUS and anal sphincter or both the EUS and pelvic floor.

Therefore, in certain embodiments of all aspects provided herein, the target pudendal nerve innervates the external urethral sphincter. In one embodiment, the target pudendal nerve does not innervate the anal sphincter or pelvic floor.

Similarly, the inventors have identified a method enabling selective modulation of the EAS such that the EUS and/or pelvic floor is/are not stimulated. The different responses induced by such selective modulation allow a high level of resolution between those pudendal branches that innervate the EAS, and those pudendal branches that innervate the EUS or pelvic floor, or those that innervate both the EAS and EUS or both the EAS and pelvic floor.

Therefore, in certain embodiments of all aspects provided herein, the target pudendal nerve innervates the external anal sphincter. In one embodiment, the target pudendal nerve does not innervate the EUS or pelvic floor.

In certain alternative embodiments of all aspects, the target pudendal nerve may innervate both the EUS and the anal sphincter or both the EUS and the pelvic floor. In this context, the inventors have determined that it is possible to apply an electrical signal to such a target pudendal nerve so as to selectively engage the EUS but not the anal sphincter or pelvic floor.

In accordance with the provided methods, if selective modulation is not detected, instead of repositioning the electrode, the electrode can be caused to apply a second stimulatory signal, where the second stimulatory signal differs from the first stimulatory signal by at least one parameter. For example the second stimulatory signal may have a different (lower or higher) frequency and/or a different (lower or higher) amplitude.

Applying a second stimulatory signal is particularly advantageous where both the first and second physiological responses have been detected beyond their respective thresholds. This indicates that the stimulated target pudendal nerve innervates more than just the EUS or just the EAS. A parameter or parameters of the second stimulatory signal can then be changed (e.g. progressively ramped up or down) and the target pudendal nerve stimulated to determine whether the first physiological response can be detected beyond its threshold when the second physiological response is not detected beyond its threshold.

That is, the parameters of the stimulatory signal can be changed such that the fibers of the target pudendal nerve innervating the target—the EUS or the EAS—are stimulated (or stimulated above the level of stimulation needed to cause the first physiological response to exceed its threshold) but the fibers of the nerve innervating non-target structures (e.g. the pelvic floor or the EAS/EUS respectively) are not stimulated (or stimulated below the level of stimulation needed to cause the second physiological response to exceed its threshold).

In certain embodiments, the amplitude of the second stimulatory signal is iteratively ramped until selective modulation is detected. In certain embodiments, the frequency of the second stimulatory signal is iteratively ramped until selective modulation is detected.

In certain embodiments of the methods, stimulating a target pudendal nerve with a second stimulatory signal is combined with repositioning the electrode to stimulate a second target pudendal nerve.

In certain embodiments of the methods of the invention, the positioning of the electrode can be optimised such that, for a given stimulatory electrical signal applied to the target pudendal nerve, preferred positioning is indicated when the first physiological response is detected (or detected to exceed the threshold) and the second physiological response is not detected (or is detected to be less than the threshold).

### Selective Modulation of the EUS

Stimulation of a pudendal nerve with parameters that selectively modulate the EUS and/or stimulation of a pudendal nerve that innervates the external urethral sphincter, and thus selectively modulates the EUS, is particularly desirable. This is especially the case for methods of treating bladder dysfunction. For example, WO2017066572 demonstrates that applying a bursting stimulation to a pudendal motor nerve is effective at promoting voiding efficiency, thereby alleviating one of the symptoms of bladder dysfunction. Bursting stimulation applied to a pudendal motor nerve will be particularly effective when applied to a pudendal nerve that innervates the external urethral sphincter.

Stimulation of a pudendal nerve innervating the EUS, especially bursting stimulation, results in a bladder voiding response, or responses indicative of bladder voiding (which includes responses indicative of imminent onset of bladder voiding). Responses indicative of onset of bladder voiding include the act of voiding itself, an increase in bladder detrusor activity (detectable by e.g. EMG), a decrease in urethral pressure, an increase in bladder contractions or a switch to pulsatile bladder contractions, and/or a decrease in bladder pressure (each measurable for example by in-dwelling catheter or other pressure sensor). Other responses indicative of bladder voiding could be those experienced and reported by the subject, for example a sensation of urgency or a sensation of EUS relaxation.

Stimulation of a pudendal nerve that innervates the EUS can also be indicated through changes in EUS activity. For example, effective bursting stimulation of such a pudendal nerve can be indicated by an increase in pulsatile EMG activity of the EUS.

By way of further example, a decrease in EUS activity in response to stimulation can be indicative of the onset of bladder voiding in response to stimulation, for example when detected in conjunction with an increase in bladder contractions. For example a decrease in EUS activity may be a decrease in EUS EMG activity or external urethral sphincter relaxation.

Similarly, stimulation of a pudendal nerve that innervates the EUS can also be indicated through changes in urethral pressure, for example, by profilometry. For example, stimulation of such a nerve can be indicated by an increase in urethral pressure, characteristic of EUS contraction in response to stimulation.

By way of further example, a decrease in urethral pressure in response to stimulation can be indicative of the onset of bladder voiding in response to stimulation, for example when detected in conjunction with an increase in bladder contractions.

Therefore, in certain preferred embodiments of all aspects when selectively modulating the EUS, the first physiological response is indicative of onset of bladder voiding.

In one particular embodiment, the first physiological response is selected from: bladder voiding, an urgency sensation, a change in EUS activity (e.g. an increase in pulsatile EUS EMG activity, a decrease in EUS EMG activity, external urethral sphincter relaxation, optionally visually-observed external urethral sphincter relaxation), an increase in bladder detrusor activity, an increase in urethral pressure, a decrease in urethral pressure, an increase in bladder contractions, an increase in bladder pulsatile contractions, and a decrease in bladder pressure, or combinations thereof.

Stimulation of a target pudendal nerve innervating the anal sphincter results in an increase in anal sphincter activity, or responses indicative of an increase in anal sphincter activity. Responses indicative of an increase in anal sphincter activity include anal sphincter contraction (detectable for example by visual inspection (observing a so-called “anal wink”) or via an in-dwelling pressure catheter), an increase in anal sphincter EMG activity, and an increase in anal sphincter pressure.

Therefore, in certain embodiments of all aspects when selectively modulating the EUS, the second physiological response is indicative of increased anal sphincter activity. In certain such embodiments, the second physiological response is selected from: an increase in anal sphincter EMG activity, an increase in anal sphincter pressure, and anal sphincter contraction, optionally visually-observed anal sphincter contraction.

In certain embodiments of all aspects, the second physiological response is indicative of increased pelvic floor activity. In such embodiments, the second physiological response is selected from: an increase in pelvic floor EMG activity, and pelvic floor contraction, optionally visually-observed contraction or contraction sensed by the subject.

In certain embodiments of all aspects, the second physiological response is indicative of increased dorsal genital nerve (DGN) activity. In such embodiments, the second physiological response is selected from: an increase in DGN neural activity, and genital sensation indicative of DGN activity and reported by the subject.

In certain embodiments of all aspects the second physiological response is indicative of increased pelvic wall muscle activity. In such embodiments, the second physiological response is selected from an increase in pelvic wall muscle EMG activity, and pelvic wall muscle twitch, for example twitch visually observed by a healthcare professional or the subject, or twitch sensed and reported by the subject.

In certain embodiments the second physiological response is any of: an increase in anal sphincter EMG activity, an increase in anal sphincter pressure, anal sphincter contraction, optionally visually-observed anal sphincter contraction, an increase in pelvic floor EMG activity, pelvic floor contraction, optionally visually-observed contraction or contraction sensed by the subject, an increase in DGN neural activity, genital sensation indicative of DGN activity reported by the subject, an increase in pelvic wall muscle EMG activity, and pelvic wall muscle twitch.

Selective modulation of the EUS is particularly effective when the stimulatory signal is a “bursting stimulation”.

Therefore, in embodiments for selectively modulating the EUS, the first and/or second stimulatory signal is a “bursting stimulation” signal as provided herein.

### Selective Modulation of the EAS

Stimulation of a pudendal nerve with parameters that selectively modulate the EAS and/or stimulation of a pudendal nerve that innervates the EAS, and thus selectively modulates the EAS, is desirable. This is especially the case for methods of treated faecal incontinence.

Stimulation of a pudendal nerve innervating the EAS results in an increase in anal sphincter activity, or responses indicative of an increase in anal sphincter activity. Responses indicative of an increase in anal sphincter activity include anal sphincter contraction (detectable for example by visual inspection (observing a so-called “anal wink”) or via an in-dwelling pressure catheter), an increase in anal sphincter EMG activity, and an increase in anal sphincter pressure.

Therefore, in certain embodiments of all aspects when selectively modulating the EAS, the first physiological response is indicative of increased anal sphincter activity. In certain such embodiments, the first physiological response is selected from: an increase in anal sphincter EMG activity, an increase in anal sphincter pressure, and anal sphincter contraction, optionally visually-observed anal sphincter contraction.

In certain preferred embodiments of all aspects when selectively modulating the EAS, the second physiological response is indicative of onset of bladder voiding. For example, the second physiological response may be one inducted by stimulation of a nerve innervating the EUS.

As noted above, stimulation of a pudendal nerve innervating the EUS can be indicated by the act of voiding itself, an increase in bladder detrusor activity (detectable by e.g. EMG), a decrease in urethral pressure, an increase in bladder contractions or a switch to pulsatile bladder contractions, and/or a decrease in bladder pressure (each measurable for example by in-dwelling catheter or other pressure sensor). Other responses indicative of bladder voiding could be those experienced and reported by the subject, for example a sensation of urgency or a sensation of EUS relaxation.

Stimulation of a pudendal nerve that innervates the EUS can also be indicated through changes in EUS activity.

By way of further example, a decrease in EUS activity in response to stimulation can be indicative of the onset of bladder voiding in response to stimulation, for example when detected in conjunction with an increase in bladder contractions. For example a decrease in EUS activity may be a decrease in EUS EMG activity or external urethral sphincter relaxation.

Similarly, stimulation of a pudendal nerve that innervates the EUS can also be indicated through changes in urethral pressure, for example, by profilometry. For example, stimulation of such a nerve can be indicated by an increase in urethral pressure, characteristic of EUS contraction in response to stimulation.

By way of further example, a decrease in urethral pressure in response to stimulation can be indicative of the onset of bladder voiding in response to stimulation, for example when detected in conjunction with an increase in bladder contractions.

Therefore, in certain preferred embodiments of all aspects when selectively modulating the EAS, the second physiological response is indicative of onset of bladder voiding.

In one particular embodiment, the second physiological response is selected from: bladder voiding, an urgency sensation, an increase in pulsatile EUS EMG activity, a decrease in EUS EMG activity, external urethral sphincter relaxation, optionally visually-observed external urethral sphincter relaxation, an increase in bladder detrusor activity, an increase in urethral pressure, a decrease in urethral pressure, an increase in bladder contractions, an increase in bladder pulsatile contractions, and a decrease in bladder pressure, or combinations thereof.

In certain embodiments, the second physiological response is indicative of increased pelvic floor activity. In such embodiments, the second physiological response is selected from: an increase in pelvic floor EMG activity, and pelvic floor contraction, optionally visually-observed contraction or contraction sensed by the subject.

In certain embodiments, the second physiological response is indicative of increased dorsal genital nerve (DGN) activity. In such embodiments, the second physiological response is selected from: an increase in DGN neural activity, and genital sensation indicative of DGN activity and reported by the subject.

In certain embodiments of all aspects the second physiological response is indicative of increased pelvic wall muscle activity. In such embodiments, the second physiological response is selected from an increase in pelvic wall muscle EMG activity, and pelvic wall muscle twitch, for example twitch visually observed by a healthcare professional or the subject, or twitch sensed and reported by the subject.

In certain embodiments the second physiological response is any of: bladder voiding, an urgency sensation, an increase in pulsatile EUS EMG activity, a decrease in EUS EMG activity, external urethral sphincter relaxation, optionally visually-observed external urethral sphincter relaxation, an increase in bladder detrusor activity, an increase in urethral pressure, a decrease in urethral pressure, an increase in bladder contractions, an increase in bladder pulsatile contractions, and a decrease in bladder pressure, an increase in pelvic floor EMG activity, pelvic floor contraction, optionally visually-observed contraction or contraction sensed by the subject, an increase in DGN neural activity, genital sensation indicative of DGN activity reported by the subject, an increase in pelvic wall muscle EMG activity, and pelvic wall muscle twitch.

In certain embodiments for selectively modulating the EAS, the first and/or second stimulatory signal is a “continuous stimulation” signal as provided herein.

Therefore, in embodiments for selectively modulating the EAS, the first and/or second stimulatory signal is a “bursting stimulation” signal as provided herein.

### Detection of Physiological Responses

Detection of the first and/or second physiological response can be performed by any suitable means, such as in-dwelling sensors (e.g. positioned by catheter or otherwise inserted or implanted), by visual inspection (either via direct observation or via imaging techniques such as ultrasound or x-ray), urethral/EUS visualization via endoscopy or cystoscopy, or by feedback reported by the subject.

In certain embodiments the first and/or second physiological responses are detected by observation by a healthcare professional or via feedback provided by the subject. In certain embodiments, the first and/or second physiological responses are detected by sensors permanently or temporarily used to monitor the physiological responses in the subject. For example the sensors may be inserted into the subject or implanted in the subject or sensors external to the subject.

In certain embodiments of all aspects, the subject is under anaesthesia for the duration of the method. In preferred such embodiments the first and second physiological responses are detected by observation by a healthcare professional, for example via visual observation or via the output from sensors inserted or implanted in the subject.

In certain embodiments, the subject is conscious for the duration of the method. In certain such embodiments the first and/or second physiological response may be detected via feedback provided by the subject, for example via reporting of a sensation of urgency or a sensation of bladder contractions, anal sphincter contractions, a pelvic floor contraction or twitch, or a genital sensation indicative of DGN engagement. The first and/or second physiological response may also be detected via feedback from the subject on the output of sensors inserted in the subject. Alternatively, in certain embodiments where the subject is conscious the first and second physiological response may be detected via observation by a healthcare professional as already described.

### Stimulatory Signal(s)

The following embodiments relate equally and independently to the first and second stimulatory signal applied in accordance with the methods of the invention. Unless otherwise specified, the embodiments relate equally and independently to the selective modulation of the EUS and to the selective modulation of the EAS.

In certain embodiments, the stimulatory electrical signal applied by the electrode comprises a waveform having a frequency in the range of from 0.1-100 Hz. In certain embodiments, the signal comprises a waveform having a frequency in the range of from 10-100 Hz. In certain preferred embodiments, the signal comprises a waveform having a frequency in the range of from 0.1-50 Hz. In certain preferred embodiments, the signal comprises a waveform having a frequency in the range of from 1-20 Hz. In certain preferred embodiments, the signal comprises a waveform having a frequency in the range of from 1-10 Hz.

In certain embodiments, bursting stimulation is applied. For “bursting stimulation” the stimulatory electrical signal applied by the electrode is applied in a burst pattern. Application of an electrical signal in a “burst pattern” refers to application of the signal in a series of bursts. That is, the signal is applied for a burst—that is, a duration of time —followed by an interval in which no signal is applied. The interval is then followed by another burst, followed by another interval in which no signal is applied. The burst pattern is the combination of the burst for which the signal is applied followed by the interval during which no interval is applied. Busting stimulation is particularly advantageous and effective for selective modulation of the EUS.

In contrast, for “continuous stimulation” there is effectively no interval between signal bursts. In such embodiments, the stimulation is applied at a given frequency uninterrupted.

In certain embodiments of all aspects where bursting stimulation is applied, the burst pattern of the stimulatory signal comprises a signal burst having a duration in the range of from 20 ms to 2000 ms, optionally in the range of from 20 ms to 500 ms, optionally a duration in the range of from 20 ms to 200 ms.

In certain preferred embodiments the burst pattern of the first electrical signal comprises a signal burst having a duration in the range of from 20 ms to 100 ms, optionally in the range of from 50 ms to 100 ms. In certain preferred embodiments the burst pattern of the first electrical signal comprises a signal burst having a duration of 50 ms. In certain preferred embodiments the burst pattern of the first electrical signal comprises a signal burst having a duration of 100 ms.

In certain embodiments, the burst pattern of the stimulatory electrical signal comprises a signal burst having a duration of from 50 ms to 1000 ms, preferably in the range of from 50 ms to 500 ms, preferably 50 ms to 200 ms, most preferably in the range of from 50 ms to 100 ms. In certain preferred embodiments, the burst pattern comprises a signal burst having a duration of 100 ms.

In certain embodiments, the burst pattern of the stimulatory electrical signal comprises a signal burst repeated at an interval of from 0.1 s to 2 s, optionally 0.125 s to 2 s. In certain embodiments, the burst pattern comprises a signal burst repeated at an interval of from 0.125 s to 1 s. In certain embodiments, the burst pattern comprises a signal burst repeated at an interval of from 0.125 s to 0.5 s. In certain alternative embodiments, the burst pattern comprises a signal burst repeated at an interval of from 0.33 s to 1 s, preferably 0.5 s to 1 s.

In certain embodiments, the burst pattern comprises a signal burst repeated at an interval of 0.125 s. In certain embodiments, the burst pattern comprises a signal burst repeated at an interval 0.2 s. In certain embodiments, the burst pattern comprises a signal burst repeated at an interval of 0.33 s. In certain embodiments, the burst pattern comprises a signal burst repeated at an interval of 0.5 s. In certain embodiments, the burst pattern comprises a signal burst repeated at an interval of 1 s.

In certain embodiments, the burst pattern of the stimulatory electrical signal comprises a signal burst having a duration from 50 ms to 1000 ms repeated at an interval of from 0.1 s to 1 s. In certain embodiments, the burst pattern comprises a signal burst having a duration from 50 ms to 1000 ms repeated at an interval of from 0.125 s to 0.5 s. In certain embodiments, the burst pattern comprises a signal burst having a duration from 50 ms to 1000 ms repeated at an interval of from 0.0.33 s to 1 s.

In certain embodiments, the burst pattern comprises a signal burst having a duration of 100 ms repeated at an interval of 0.125 s. In certain embodiments, the burst pattern comprises a signal burst having a duration of 100 ms repeated at an interval of 0.2 s. In certain embodiments, the burst pattern comprises a signal burst having a duration of 100 ms repeated at an interval of 0.5 s. In certain embodiments, the burst pattern comprises a signal burst having a duration of 100 ms repeated at an interval of 1 s.

In certain embodiments, the burst pattern of the stimulatory electrical signal consists of a signal burst having a duration from 50 ms to 1000 ms repeated at an interval of from 0.1 s to 1 s. In certain embodiments, the burst pattern consists of a signal burst having a duration from 50 ms to 1000 ms repeated at an interval of from 0.125 s to 0.5 s. In certain embodiments, the burst pattern consists of a signal burst having a duration from 50 ms to 1000 ms repeated at an interval of from 0.0.33 s to 1 s.

In certain embodiments, the burst pattern consists of a signal burst having a duration of 100 ms repeated at an interval of 0.125 s. In certain embodiments, the burst pattern consists of a signal burst having a duration of 100 ms repeated at an interval of 0.2 s. In certain embodiments, the burst pattern consists of a signal burst having a duration of 100 ms repeated at an interval of 0.5 s. In certain embodiments, the burst pattern consists of a signal burst having a duration of 100 ms repeated at an interval of 1 s.

In some embodiments, the stimulatory electrical signal applied in a burst pattern comprises a signal burst repeated at a frequency in the range of from 0.5 to 20 Hz. For example, a burst pattern comprising a signal burst at 40 Hz repeated at a frequency of 2 Hz would repeat the signal burst at 0.5 s intervals (see diagram below).

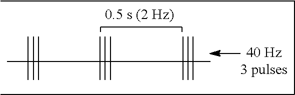

In some embodiments, the stimulatory electrical signal applied in a burst pattern comprises a signal burst repeated at a frequency in the range of 0.5 to 20 Hz. In some embodiments, the stimulatory electrical signal applied in a burst pattern comprises a signal burst repeated at a frequency in the range of 1 to 10 Hz, preferably in the range of from 1 Hz to 8 Hz. In some embodiments, the stimulatory electrical signal applied in a burst pattern comprises a signal burst repeated at a frequency in the range of from 1 Hz to 5 Hz, preferably from 1 Hz to 3 Hz, more preferably from 1 to 2 Hz.

In one embodiment, the stimulatory electrical signal comprises a signal burst repeated at a frequency comprising 10 Hz, optionally wherein the stimulatory electrical signal comprises an AC waveform repeated at a frequency of 10 Hz. In another embodiment, the electrical signal is repeated at a frequency comprising 1 Hz, optionally wherein the electrical signal is an AC waveform repeated at a frequency of 1 Hz. In another embodiment, the electrical signal is repeated at a frequency comprising 2 Hz, optionally wherein the electrical signal is an AC waveform repeated at a frequency of 2 Hz. In another embodiment, the electrical signal is repeated at a frequency comprising 3 Hz, optionally wherein the electric signal is an AC waveform repeated at a frequency of 3 Hz.

In another embodiment, the electrical signal is repeated at a frequency comprising 4.76 Hz, optionally wherein the electric signal is an AC waveform repeated at a frequency of 4.76 Hz. In another embodiment, the electrical signal is repeated at a frequency comprising 8 Hz, optionally wherein the electric signal is an AC waveform repeated at a frequency of 8 Hz.

In certain embodiments, the stimulatory electrical signal comprises a signal burst wherein the signal burst comprises a waveform having a frequency in the range of from 10-100 Hz. In certain embodiments, the stimulatory electrical signal comprises a signal burst wherein the signal burst comprises a waveform having a frequency in the range of from 20-50 Hz. In certain embodiments, the signal burst comprises a waveform having a frequency in the range of from 30-50 Hz, optionally in the range of from 30-40 Hz. In certain such embodiments, the signal burst has a frequency of 40 Hz.

In some embodiments, the burst pattern consists of from 1 to 10 pulses per signal burst. In some embodiments, the burst pattern consists of from 1 to 5 pulses per signal burst. In some embodiments, the burst pattern consists of 3 pulses per signal burst. In such embodiments, the duration of the signal burst is thus determined by the frequency of the waveform.

In certain embodiments where continuous stimulation is applied, the stimulatory electrical signal comprises a waveform having a frequency in the range of from 10-100 Hz. In certain embodiments, the stimulatory electrical signal comprises a waveform having a frequency in the range of from 1 Hz to 50 Hz, optionally from 20 Hz to 50 Hz. In certain embodiments, the signal comprises a waveform having a frequency in the range of from 1-40 Hz, optionally in the range of from 1-20 Hz. In certain embodiments the signal comprises a waveform having a frequency of 10 Hz or 20 Hz.

In certain embodiments, the signal comprises a waveform having a frequency in the range of from 30-50 Hz, optionally in the range of from 30-40 Hz. In certain such embodiments, the signal has a frequency of 40 Hz.

In certain embodiments, the stimulatory electrical signal applied by the electrode has an amplitude in the range 0.1 to 10 T.

“T” is a measure of relative stimulation intensity. Relative stimulation intensity can be expressed as multiples (0.1, 0.8, 1, 2, 5, etc.) of “T”. “T” represents the threshold stimulation intensity to evoke a motor response. For example, “1 T” is defined as the threshold stimulation intensity required to evoke a motor response—in particular, as used herein “T” may be defined as the threshold amplitude required to evoke a reflex response in the external urethral sphincter (EUS) (as determined by electromyogram (EMG) or EUS pressure measurements) when the electrical signal is applied to the pudendal nerve.

Determining “T” as described herein provides a calibration baseline able to be transferred between subjects and/or species. T thus provides a useful measure for amplitude normalization between subjects and/or species. For example, T may be determined as follows: a low frequency electrical signal (e.g., 1 Hz) is applied and the intensity of stimulation is increased (either by increasing the voltage or the current of the signal, preferably the current) until the pudendal nerve stimulation produces a reflex EMG response in the EUS. This stimulation intensity is designated T. The absolute threshold stimulation intensity may vary across subjects and/or species due to inherent variation, positioning and type of the electrode, etc., and therefore subsequent experimental or therapeutic intensities are designated as multiples of T to provide equivalent relative stimulation intensities.

The desired stimulation intensity (i.e. the desired multiple of threshold intensity “T”) can be achieved through controlled variation of the current or voltage of the signal, preferably the current.

In such embodiments, the stimulatory electrical signal has an amplitude in the range from 0.05 T to 5.0 T. In certain embodiments, the signal has an amplitude in the range from 0.3 T to 3 T. In some embodiments, the electrical signal has an amplitude of 0.05, 0.1, 0.2, 0.3, 0.4, 0.5 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 T.

In certain embodiments, the stimulatory electrical signal has an amplitude value of from 0.5 T to 3 T, preferably in the range of from 1 T to 3 T. In some embodiments, the electrical signal has a T value of 1 T, 1.5 T, 1.8 T 2 T, 2.3 T, 2.5 T or 3 T. In certain preferred embodiments the electrical signal has an amplitude in the range of from 1.8 T to 2.3 T.

In other embodiments, the stimulatory electrical signal has an amplitude of 1 T. In other embodiments, the electrical signal has an amplitude of 1.5 T. In other embodiments the electrical signal has an amplitude of 2 T.

In other embodiments, the stimulatory electrical signal has an amplitude in the range of from 0.1-20 mA, optionally 0.1-15 mA, optionally 0.1-10 mA, optionally 0.1-5 mA. In certain embodiments, the stimulatory electrical signal has an amplitude in the range of from 5-20 mA. In other embodiments, the stimulatory electrical signal has an amplitude in the range of from 5-15 mA. In certain embodiments, the stimulatory electrical signal has an amplitude in the range of from 5-10 mA. In certain such embodiments the stimulatory electrical signal has an amplitude in the range of from 1-5 mA, optionally 1 mA, 2 mA, 3 mA, 4 mA or 5 mA.

In other embodiments, the stimulatory electrical signal has an amplitude in the range of from 0.1-1 mA, optionally 0.2-1 mA. In certain embodiments the signal has an amplitude of 0.6 mA-1 mA.

In certain embodiments the signal has an amplitude of 100-500 μA, optionally 100-400 μA. In certain embodiments, the electrical signal has an amplitude of 100 μA, 200 μA, or 400 μA.

It will be appreciated by the skilled person that an electronic signal “comprising” an indicated frequency may have other frequency components as part of the signal. In certain preferred embodiments of all aspects where a signal comprises an indicated frequency, the indicated frequency is the dominant frequency component of the signal.

In certain embodiments, the stimulatory signal comprises a DC waveform.

In certain embodiments, the stimulatory signal is monophasic. In certain embodiments, the stimulatory signal is biphasic.

In certain embodiments, the stimulatory signal is an AC waveform, optionally biphasic AC waveform, optionally a charge-balanced biphasic AC waveform. In certain such embodiments, the waveform may be symmetrical or asymmetrical.

In certain embodiments, each phase of the biphasic waveform has a phase duration from 0.005 ms to 2 ms, optionally 0.01 to 1 ms, optionally 0.05 to 0.5 ms, optionally 0.05 to 0.2 ms, optionally 0.1 ms. In certain embodiments, each phase of a biphasic waveform is of equal duration. In certain alternative embodiments, each phase is of a different duration.

The AC waveform may be selected from sinusoidal, triangular, square or a complex waveform.

Once selective modulation of the EUS or EAS has been achieved as a result of a method provided herein, it may be desirable to optimise the parameters of the electrical signal to be applied by the electrode. For example, parameters such as the amplitude, frequency, pulse width and/or pattern (for example, a burst pattern) of the electrical signal could be modulated in order that application of the electrical signal by the electrode achieves the desired physiological response in the subject but minimises the discomfort experienced by the subject during application of the electrical signal.

Therefore, in certain embodiments, once selective modulation of the EUS or EAS has been detected, the method further comprises the step of modulating at least one parameter of the electrical signal applied by the electrode, for example to optimise efficacy or patient comfort, wherein the at least one parameter of the electrical signal is selected from amplitude, frequency and burst pattern.

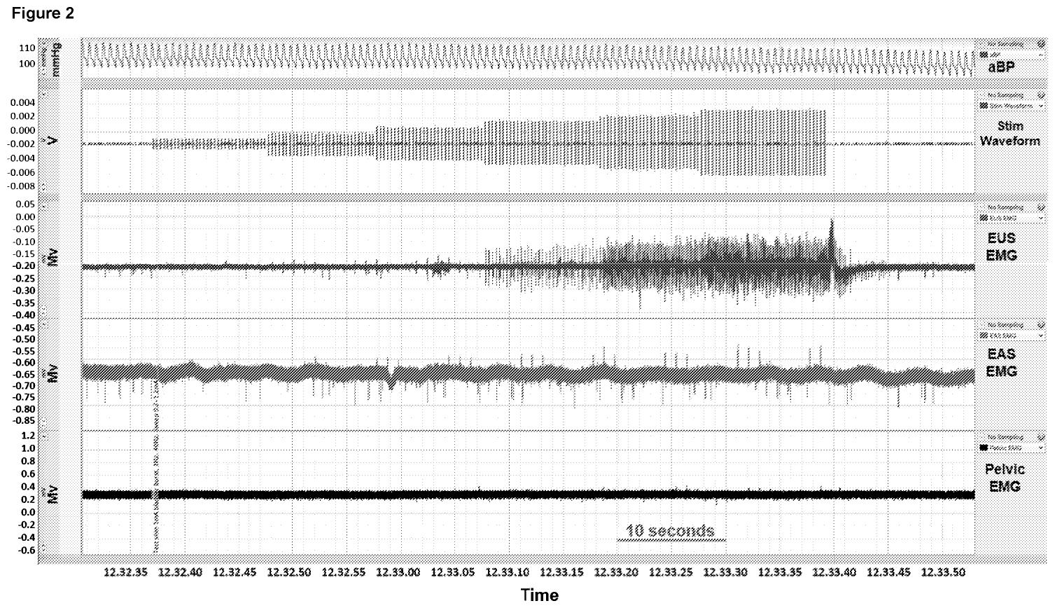

It has been demonstrated herein that once selective modulation of the EUS or EAS has been detected, it is possible to increase the amplitude of the stimulatory electrical signal without losing the selectivity (FIG. 2). For example, the signal amplitude can be increased without increasing pelvic muscle EMG or EAS EMG activity. This is advantageous as it allows the strength of the effect on the EUS to be modulated (e.g. to improve efficacy) without increasing nonspecific stimulation effects.

Accordingly, in certain embodiments of all aspects, the method further comprises the step of modulating (e.g. increasing) the amplitude of the stimulatory signal (e.g. the therapeutic stimulatory signal). Preferably the modulation in amplitude benefits the subject, for example by improving comfort and/or efficacy of treatment.

In preferred embodiments of all aspects, the electrode is part of a neurostimulation apparatus. In such embodiments, the apparatus comprises an electrode and a controller coupled to the electrode and controlling the stimulatory electrical signal to be applied by the electrode, wherein the controller is configured to cause the electrode to apply the stimulatory electrical signal. Suitable apparatuses for stimulation of a pudendal nerve in a subject are described in WO2017066572. The apparatuses and embodiments thereof described in WO2017066572 are incorporated herein by reference in their entirety.

In certain embodiments of the methods provided herein, the method further comprises programming the neurostimulation apparatus comprising the electrode to apply the stimulatory electrical signal to the target pudendal nerve.

In certain embodiments, the apparatus comprises at least one detector configured to detect the first physiological response. In certain embodiments the apparatus comprises at least one detector configured to detect the second physiological response. In certain such embodiments, the controller of the apparatus is configured to follow the steps of a method provided herein.

It is a further advantage of the present disclosure to provide a neurostimulation apparatus comprising an electrode, where the apparatus can evaluate the positioning of the electrode in accordance with a method provided herein.

Therefore in a further aspect is provided a neurostimulation apparatus comprising at least one electrode and a controller coupled to the electrode, wherein the controller is configured to follow the steps of a method provided herein.

In certain embodiments, the apparatus further comprises at least one detector configured to detect the first physiological response. In certain embodiments the apparatus further comprises at least one detector configured to detect the second physiological response.

In certain embodiments, the apparatus further comprises a user input interface such that the subject and/or a healthcare professional can enter the outcome of their detection of the first and/or second physiological response.

In certain embodiments, the apparatus further comprises a user display interface configured to report the outcome of the evaluation of the electrode positioning to the subject and/or a healthcare professional.

The provided methods and apparatuses will be particularly advantageous for use in the context of treating bladder dysfunction in a subject, for example in treating underactive bladder (UAB) and/or urinary retention (UR).

For example, as part of a therapeutic method for treating bladder dysfunction which involves the stimulation of a target pudendal nerve (e.g. to increase voiding efficiency), the positioning of and/or signal applied by the electrode for stimulating the target pudendal nerve may first be evaluated according to a method provided herein such that selective modulation of the EUS is achieved. Doing so will increase the efficacy of the therapeutic method by ensuring the stimulation is accurately applied to the target pudendal nerve.

Suitable therapeutic methods for treating bladder dysfunction (e.g. overactive bladder (OAB) or underactive bladder (UAB)) by pudendal nerve stimulation are provided in WO2017066572, and WO2019023115 each of which is incorporated herein by reference in its entirety. It is particularly preferred to use the provided methods and apparatuses in methods of treating bladder dysfunction by applying a bursting stimulation to a pudendal motor nerve, as described in WO2017066572.

As provided herein, the methods and apparatuses will also be particularly advantageous for use in treatment of faecal incontinence.

The term “patient” where used herein is used interchangeably with “subject”. In a preferred embodiment of all aspects, the subject is a human subject.

### Examples

The following specific method is provided as a non-limiting example of the methods set out herein:
(i) A subject is identified as suffering from bladder dysfunction and in need of treatment by pudendal nerve stimulation—specifically, stimulation of a pudendal motor nerve innervating the EUS such that the EUS is selectively modulated;
(ii) Laparoscopic techniques are used to visualise anatomical landmarks in the subject such as the pudendal canal, and the pudendal nerve and its branching points;
(iii) Using the anatomical landmarks, a determination is made as to the location of a pudendal motor nerve innervating the EUS;
(iv) A guide lead is inserted into the subject to position an electrode such that the electrode is intended to stimulate the identified pudendal motor nerve;
(v) A stimulatory electrical signal is applied through the positioned electrode;
(vi) The EUS EMG response and the anal sphincter EMG response are detected;
(vii) If both EUS and anal sphincter show an EMG response greater than an unstimulated (natural) baseline level of EMG activity, or no EUS EMG response is detected relative to the baseline activity, the guide lead is used to reposition the electrode and the stimulatory electrical signal is reapplied;
(viii) If an EUS EMG response greater than the baseline level of activity is detected in reaction to the stimulation and no anal sphincter EMG response greater than baseline is detected, the electrode is evaluated as positioned for selective modulation of the EUS;
(ix) The electrode is then left in its position coupled to a controller, where the controller is configured such that it will apply a stimulation protocol via the electrode in accordance with the intended method of treatment.

The following specific method is provided as a further non-limiting example of the methods set out herein:
(i) A subject is identified as suffering from bladder dysfunction and in need of treatment by pudendal nerve stimulation—specifically, stimulation of a pudendal motor nerve innervating the EUS;
(ii) Laparoscopic techniques are used to visualise anatomical landmarks in the subject such as the pudendal canal, and the pudendal nerve and its branching points;
(iii) Using the anatomical landmarks, a determination is made as to the location of a pudendal motor nerve innervating the EUS and the anal sphincter;
(iv) A guide lead is inserted into the subject to position an electrode such that the electrode is intended to apply an electrical signal to the identified pudendal motor nerve so as to selectively modulate the EUS and not the anal sphincter;
(v) The selectively stimulatory electrical signal is applied through the positioned electrode;
(vi) The EUS pressure response and the anal sphincter contraction response are detected;
(vii) If no EUS pressure response is detected, the guide lead is used to reposition the electrode and the stimulatory electrical signal is reapplied and step (vi) repeated;
(viii) If both an anal wink and an EUS pressure response is detected, the frequency of the stimulatory signal is varied and steps (v)-(vi) repeated;
(ix) If an EUS pressure response is detected in reaction to the stimulation and no anal wink is detected, the electrode position and stimulatory signal are suitable for selective modulation of the EUS;
(x) Once the position has been determined using the guide lead, an electrode, for example, a cuff electrode, is then placed in the position, the electrode being coupled to a controller, where the controller is configured such that it will apply a stimulation protocol via the electrode in accordance with the intended method of treatment of bladder dysfunction.

The following specific method is provided as a further non-limiting example of the methods set out herein:
(i) A subject is identified as suffering from faecal incontinence and in need of treatment by pudendal nerve stimulation—specifically, pudendal nerve stimulation for selective modulation of the EAS;
(ii) Laparoscopic techniques are used to visualise anatomical landmarks in the subject such as the pudendal canal, and the pudendal nerve and its branching points;
(iii) Using the anatomical landmarks, a determination is made as to the location of a pudendal motor nerve innervating the EAS and the EUS;
(iv) A guide lead is inserted into the subject to position an electrode such that the electrode is intended to apply an electrical signal to the identified pudendal motor nerve so as to selectively stimulate the EAS and not the EUS;
(v) The selectively stimulatory electrical signal is applied through the positioned electrode;
(vi) The EAS contraction response (via endoscopy) and the EUS EMG response are detected;
(vii) If no EAS contraction response is detected, the guide lead is used to reposition the electrode and the stimulatory electrical signal is reapplied and step (vi) repeated;
(viii) If both EAS contraction (“anal wink”) and an EUS EMG response is detected, the frequency of the stimulatory signal is varied and steps (v)-(vi) repeated;
(ix) If an EAS contraction response is detected in reaction to the stimulation and no EUS response is detected, the electrode position and stimulatory signal are suitable for selective modulation of the EAS;
(x) Once the position has been determined using the guide lead, an electrode, for example, a cuff electrode, is then placed in the position, the electrode being coupled to a controller, where the controller is configured such that it will apply a stimulation protocol via the electrode in accordance with the intended method of treatment of faceal incontinence.

### In Vivo Example

Objective

In anaesthetised models there is an inherent retention associated with anaesthesia resulting in higher post void residual (PVR) volumes, where voiding efficiency is reduced to 10-40%. Application of bursting stimulation to the pudendal motor (perineal) nerve in preclinical efficacy experiments during voiding promoted the rhythmic opening and closing of the external urethral sphincter (EUS) and bladder outlet to produce a pulsatile flow of urine, activating afferent sensory feedback, to elicit bladder contractions. Data from two species (rats and cats) demonstrated that burst stimulation (shown schematically below—2 Hz bursts, 40 Hz, 100 ms duration, and 3 (or 4) pulses per burst) on the pudendal motor nerve during micturition produces large increases in voiding efficiency. This offers a novel approach for treating bladder dysfunction, for example underactive bladder (UAB), where pharmacological and neuromodulation interventions have been ineffective.

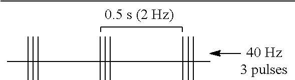

The aim of this study was to assess whether target engagement (i.e. pulsatile activation of the EUS) is possible in an animal with similarly sized nerve structures and EUS to humans. Gross pudendal nerve anatomy studies determined the sheep as most appropriate, where branching and surrounding structures were most similar to humans.

Methods

Three sheep were used in this study (IDs: 322, 323, 347). The sheep were anaesthetised using ketamine (5 mg/kg) and midazolam (0.5 mg/kg) injected IV. A tracheal tube was inserted into the trachea for the primary purpose of establishing and maintaining a patent airway and to maintain general anaesthesia using sevoflurane carried in an oxygen/air mixture. After induction of general anaesthesia, the animal was positioned in dorsal recumbency. Indwelling catheters were percutaneously placed in both the external jugular veins and one in the femoral artery (for blood pressure and blood gas monitoring) using ultrasonographic guidance. An alternative arterial line was placed in the left or right ear if required. An intra-oesophageal tube was inserted to drain refluxes from the rumen.

Palpebral reflex, corneal reflex, medioventral eye ball position, jaw tone was used to monitor anaesthetic depth. Nystagmus as well as lacrimation was monitored as possible signs of light plane of anaesthesia. Electrocardiogram (ECG), Heart rate (HR), arterial blood pressure, respiratory rate (RR), pulse oximetry, capnography, spirometry, body temperature were monitored throughout the surgery. Body temperature was recorded continuously with an intranasal probe. Arterial blood gasses were analysed throughout the experiment to monitor pH, Glucose, PaO2 and PaCO2, K+ levels. The depth of anaesthesia was assessed by looking at physiological response as well as using a bispectral index monitoring system (levels between 30 and 60). Levels of sevoflurane were adjusted accordingly by the anaesthetist.

The sheep were placed into lithotomy position and the appropriate draping positioned. The ischial tuberosity and pubic symphysis palpated, and an incision approximately 4 cm long made cephalad to the ischial tuberosity, just lateral to the vulva. The ischiorectal fossa was then entered through this incision. Hemostasis was obtained throughout with a bipolar coagulator set at the lowest energy level consistent with obtaining coagulation. A small Weitlander retractor was used to spread more deeply into the ischiorectal fossa looking for the pudendal perineal branches that travel transversely to the incision; this involved smooth tissue dissection with scissors. From cadaver dissections there are often 2 or 3 branches, unless the perineal nerve divides quite distally, in which case the one branch will be much larger.

Once pudendal nerves were visualized, electromyography (EMG) leads were placed in the EUS muscle, pelvic floor muscle, and external anal sphincter (EAS) to monitor selective/non-selective muscle activity during periods of pudendal nerve stimulation. Urethral pressure (Medica S.p.A Pico urodynamic system, Genesis Medical Ltd) was monitored with the use of a two-lumen 8 F catheter placed via natural routes in the urethra; the two side-holes of this catheter in contact with the urtheral sphincter and in the bladder, respectively. Flow profilometry with a slow rate of 2 mL/min also require a 10 F Foley catheter in place in the bladder via the urethra.

Initially, to identify the individual pudendal branching innervations a point stimulator was used to sequentially stimulate the differing pudendal nerves whilst monitoring EMG signals to determine whether selective engagement of the EUS, EAS or pelvic floor was evident. The EUS was also visualized to assess nerve target engagement via an endoscope. The endoscope was passed into the urethra to the base of the bladder neck, monitoring urethral sphincter contraction and relaxation during periods of stimulation. Once EUS activation was confirmed, an Ad-Tech Spencer Probe depth electrode was then placed adjacent to the selected pudendal motor nerves to assess whether biphasic bursting electrical stimulation (Digitimer DS5 clinical grade stimulator) could recruit a sufficient amount of the smaller motor fibers to confirm target EUS engagement without activating pelvic floor or the EAS. Signals were recorded using PowerLab (AD instruments). Stimulations (0.1 ms pulse width, 0.01 ms Interphase interval) were increased in 1 mA steps to assess EUS activation. Initially, 0.5 s (2 Hz) interburst intervals were tested, before exploring the effects of other interburst intervals, e.g. 1 Hz, 3 Hz, 8 Hz on EUS activation.

Finally, in addition to the surgically open access method and placement of the Adtech interface on the pudendal nerves, a closed placement of the percuataneous lead was attempted. Ultrasound was used to assess structures and vessels for placement, and an introducer used to estimate the depth to pudendal nerves on the open side with finger guidance to place the electrode on the closed side. Stimulation was initiated at 1 mA, ramping up to measure EUS engagement, with smaller increments used as required.

Results

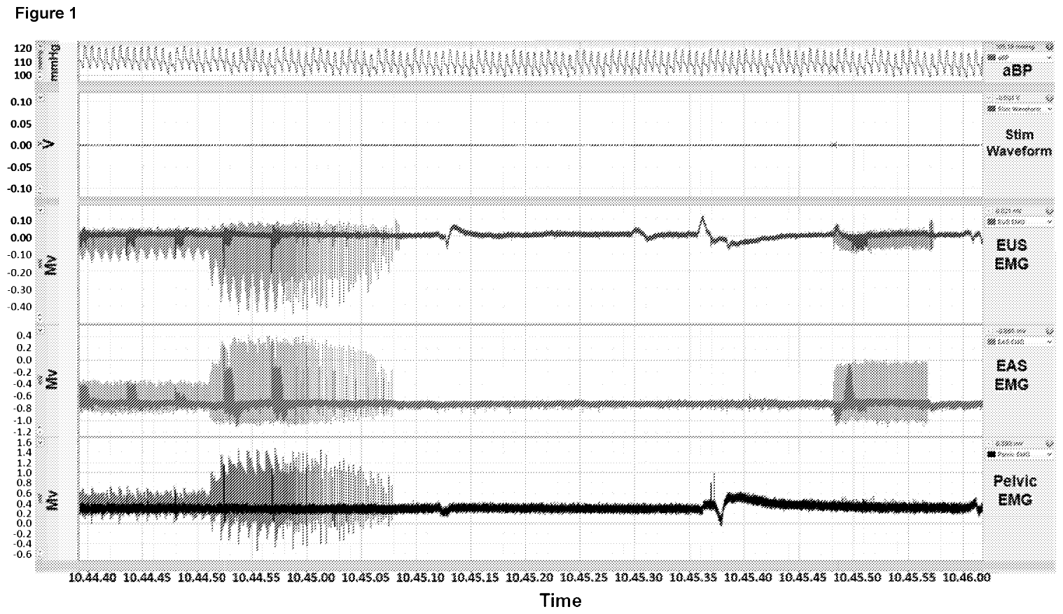

Following open dissection of the left side lateral to the vulva, 3-4 pudendal branches were identified in all three sheep, however, end target innervation was unknown. Using the point stimulator and parameter scoping (5-10 mA; 10-20 Hz; 200 s; monophasic) across each of the branches differing target engagement profiles were evident. As an example of non-selective effects in sheep ID 322, stimulation on Branch 1 confirmed urethral sphincter contraction using endoscopic visualization that ceased once stimulation stopped. Anal and vaginal wall twitching responses occurred with stimulation, and EMG readings were altered by moving the point stimulator along the pudendal branch. EMGs initially contained stimulation artifact but activity correlated with visual observations of non-selective EMG engagement via endoscopic EUS monitoring, anal twitch, and pelvic muscle movement. At the initial stimulation point on Branch 1 there was a clear non-selective engagement of the EUS, EAS, and pelvic muscle. However, moving stimulation along the nerve altered the response, to selectively activate the EUS and EAS (FIG. 1).

Using this approach enabled identification of the most appropriate nerve branch to assess the effects of biphasic bursting stimulation via the Adtech interface (containing 4 contact points) and selective EUS modulation. For example, in sheep 323 stimulation at 5 mA on the selected branch produced a contraction of EUS muscle with associated EMG activity. An amplitude sweep (1 mA increments) from 1-6 mA for 10 seconds at each step produced an increase in urethral sphincter pressure from 5 cmH₂O (baseline) to 32 cmH₂O at 1 mA, reaching 37 cmH₂O at 6 mA. Some anal pressure increases EAS EMG spikes were observed. Due to the large increase in sphincter pressures observed at 1 mA, lower amplitudes were then explored (0.2-1 mA) in 0.2 mA increments. Urethral sphincter pressure increased from a baseline of 5 to 14 cmH₂O at 0.6 mA, 25 cmH₂O at 0.8 mA, and 29 cmH₂O at 1 mA. This demonstrated a clear elective effect of increasing amplitude on the urethral pressure, that correlated with EUS EMG activity, without affecting the EAS or pelvic muscle (FIGS. 2 & 3) on this pudendal branch.

The effects of differing interburst intervals at 2 mA was explored using endoscopic visualization of the EUS to determine whether pulsatile/rhythmic opening and closing could be induced with bursting stimulation. 2 mA/8 Hz bursting stimulation produced a full sustained EUS contraction and closure of the bladder neck, whereas, 2 mA/2 Hz bursting stimulation resulted in the desired phasic pulsatile EUS contraction and relaxation. Across the sheep, pulsatile activity was observed at interphase intervals of 1, 2, and 3 Hz, whereas 8 Hz induced contraction only. This suggests 8 Hz frequency is too high to elicit pulsatile EUS engagement in a sphincter representative of human sizing—compared to prior data in rats where 8 Hz elicited phasic effects.

In addition to the effects of stimulation on the defined pudendal branches, selective activation of the EUS via the Adtech interface was possible with percutaneous placement. In sheep 347 an introducer was used to estimate the depth and distance from the surface to the pudendal structures on the open side (4 cm). Finger guidance in the vagina was used to place the needle introducer in the closed side along the vaginal wall to a depth of 4 cm, where the Adtech electrode was then placed. Ramping stimulation to 4.5 mA produced a visual vaginal wall contraction on the open side with associated EUS EMG activity. Combinations of the 4 electrode contacts were then tested at 5 mA, producing reproducible EUS activation using contacts 1&4:

TABLE 1

| Contacts tested | Observation |
|---|---|
| 1&4 | EUS EMG |
| 3&4 | No effects |
| 1&4 repeat | EUS EMG |
| 1&2 | No effects |
| 2&3 | No effects |
| 3&4 | No effects |
| 1&4 repeat | EUS EMG (reconfirming activity without |
| | recruitment on pelvic muscle or EAS) |

Finally, EUS activation was further confirmed with endoscopy, demonstrating a clear pulsatile urethral contraction and relaxation to bursting stimulation.

### SUMMARY

Open assessment demonstrated that reproducible selective EUS activation could be achieved without recruitment of other muscles (pelvic, EAS).
Furthermore, closed percutaneous perineal placement at a depth of 4 cm using bursting stimulation also successfully produced selective EUS activity without off-target recruitment.
Gross visual examination (including ultrasound) of the pudendal branching was not sufficient to identify end target innervations points. Pelvic muscle, EUS, and EAS activation could only be confirmed through selective nerve stimulation to identify to the target interfacing site. This was demonstrated by sequentially exploring the innervation points with the associated on-/off-target physiological measures, and also by altering the stimulation fields (e.g. narrow or broad).
Compared to prior small animal efficacy studies (rat and cat) where differing bursting paradigms (2 Hz, 4.76 Hz, and 8 Hz) have been efficacious through eliciting rhythmic EUS contraction/relaxation, the larger EUS in sheep (and by extension humans) functions differently to selected parameters.
   8 Hz bursting was shown to induce a sustained EUS contraction as the larger EUS muscle is unable to rhythmically contract/relax at such high frequencies.
   However, the phasic effects of bursting stimulation were observed at 1 Hz, 2 Hz and 3 Hz in this model which provided sufficient interphase periods for muscle contraction/relaxation that would also be relevant to humans.

1. A method of selectively modulating an external urethral sphincter (EUS) or external anal sphincter (EAS) of a subject, the method comprising:
(a) positioning an electrode for stimulating a first target pudendal nerve in a subject, wherein the electrode is configured to apply a stimulatory electrical signal to stimulate a target pudendal nerve;
(b) causing the electrode to apply a first stimulatory electrical signal, such that the electrode stimulates the first target pudendal nerve;
(c) detecting a magnitude of a first physiological response and a magnitude of a second physiological response in response to the first stimulatory electrical signal applied by an electrode;
(d) detecting a presence or absence of selective modulation of the EUS or EAS, wherein:
i. selective modulation of the EUS or EAS is absent when the magnitude of the first physiological response is detected to be not beyond a first predetermined threshold and/or the magnitude of the second physiological response is detected to be beyond a second predetermined threshold;
ii. selective modulation of the EUS or EAS is present when the first physiological response is detected to be beyond the first predetermined threshold and the second physiological response is detected to be not beyond the second predetermined threshold;
(e) if selective modulation of the EUS or EAS is detected to be absent:
iii. re-positioning the electrode to stimulate a second target pudendal nerve; and/or
iv. causing the electrode to apply a second stimulatory signal, such that the electrode stimulates the target pudendal nerve, where the second stimulatory signal differs from the first stimulatory signal by at least one parameter; and
(f) repeating steps (a)-(e) until the selective stimulation of the EUS or EAS is detected as present.

2. A method of treating a subject with bladder dysfunction by selective modulation of an EUS, the method comprising:
(a) identifying a first target pudendal nerve in a subject who has been diagnosed with bladder dysfunction:
(b) positioning an electrode for stimulating the first target pudendal nerve in the subject, wherein the electrode is configured to apply a stimulatory electrical signal to stimulate a target pudendal nerve;
(c) causing the electrode to apply a first stimulatory electrical signal, such that the electrode stimulates the first target pudendal nerve;
(d) detecting a magnitude of a first physiological response and a magnitude of a second physiological response in response to the first stimulatory electrical signal applied by an electrode;
(e) detecting a presence or absence of selective modulation of the EUS, wherein:
i. selective modulation of the EUS is absent when the magnitude of the first physiological response is detected to be not beyond a first predetermined threshold and/or the magnitude of the second physiological response is detected to be beyond a second predetermined threshold;
ii. selective modulation of the EUS is present when the first physiological response is detected to be beyond the first predetermined threshold and the second physiological response is detected to be not beyond the second predetermined threshold;
(f) if selective modulation of the EUS is detected to be absent:
iii. re-positioning the electrode to stimulate a second target pudendal nerve; and/or
iv. causing the electrode to apply a second stimulatory signal, such that the electrode stimulates the target pudendal nerve, where the second stimulatory signal differs from the first stimulatory signal by at least one parameter;
(g) repeating steps (a)-(f) until the selective modulation of the EUS is detected as present; and
(h) treating the bladder dysfunction in the subject by causing an electrode to apply a stimulatory signal, where the electrode is at the position in step (g) that resulted in selective modulation of the EUS.

3. A method of treating a subject with faecal incontinence by selective modulation of an EAS, the method comprising:
(a) identifying a first target pudendal nerve in a subject who has been diagnosed with faecal incontinence:
(b) positioning an electrode for stimulating the first target pudendal nerve in the subject, wherein the electrode is configured to apply a stimulatory electrical signal to stimulate a target pudendal nerve;
(c) causing the electrode to apply a first stimulatory electrical signal, such that the electrode stimulates the first target pudendal nerve;
(d) detecting a magnitude of a first physiological response and a magnitude of a second physiological response in response to the first stimulatory electrical signal applied by an electrode;
(e) detecting a presence or absence of selective modulation of the EAS, wherein:
i. selective modulation of the EAS is absent when the magnitude of the first physiological response is detected to be not beyond a first predetermined threshold and/or the magnitude of the second physiological response is detected to be beyond a second predetermined threshold;
ii. selective modulation of the EAS is present when the first physiological response is detected to be beyond the first predetermined threshold and the second physiological response is detected to be not beyond the second predetermined threshold;
(f) if selective modulation of the EAS is detected to be absent:
iii. re-positioning the electrode to stimulate a second target pudendal nerve; and/or
iv. causing the electrode to apply a second stimulatory signal, such that the electrode stimulates the target pudendal nerve, where the second stimulatory signal differs from the first stimulatory signal by at least one parameter;
(g) repeating steps (a)-(f) until the selective modulation of the EAS is detected as present; and
(h) treating the faecal incontinence in the subject by causing an electrode to apply a stimulatory signal, where the electrode is at the position in step (f) that resulted in selective modulation of the EAS.

4. (canceled)

5. The method according to claim 1 when the method is a method for selectively modulating the EUS, wherein the first physiological response is selected from: bladder voiding, an urgency sensation, a change in EUS activity (e.g. an increase in pulsatile EUS EMG activity, a decrease in EUS EMG activity, external urethral sphincter relaxation, optionally visually-observed external urethral sphincter relaxation), an increase in bladder detrusor activity, an increase in urethral pressure, a decrease in urethral pressure, an increase in bladder contractions, an increase in bladder pulsatile contractions, and a decrease in bladder pressure, or combinations thereof.

6. (canceled)

7. The method according to claim 1, wherein the second physiological response is indicative of increased anal sphincter activity, pelvic floor activity, dorsal genital nerve (DGN) activity or pelvic wall muscle activity and is selected from: an increase in anal sphincter EMG activity, an increase in anal sphincter pressure, and anal sphincter contraction, optionally visually-observed anal sphincter contraction, an increase in pelvic floor EMG activity, pelvic floor contraction, optionally visually-observed contraction or contraction sensed by the subject, an increase in DGN neural activity, genital sensation indicative of DGN activity and reported by the subject, an increase in pelvic wall muscle EMG activity, and pelvic wall muscle twitch.

8. The method according to claim 1 when the method is a method for selectively modulating the EUS wherein the target pudendal nerve is a perineal nerve.

9. The method according to claim 1 when the method is a method for selectively modulating the EUS wherein the target pudendal nerve innervates the external urethral sphincter.

10. The method according to claim 1 when the method is a method for selectively modulating the EAS, wherein the first physiological response is selected from: anal sphincter contraction, an increase in anal sphincter EMG activity, and an increase in anal sphincter pressure.

11. (canceled)

12. The method according to claim 1, wherein the second physiological response is indicative of increased EUS activity, pelvic floor activity, dorsal genital nerve (DGN) activity, or pelvic wall muscle activity and is selected from: bladder voiding, an urgency sensation, an increase in pulsatile EUS EMG activity, a decrease in EUS EMG activity, external urethral sphincter relaxation, optionally visually-observed external urethral sphincter relaxation, an increase in bladder detrusor activity, an increase in urethral pressure, a decrease in urethral pressure, an increase in bladder contractions, an increase in bladder pulsatile contractions, and a decrease in bladder pressure, an increase in pelvic floor EMG activity, pelvic floor contraction, optionally visually-observed contraction or contraction sensed by the subject, an increase in DGN neural activity, genital sensation indicative of DGN activity reported by the subject, an increase in pelvic wall muscle EMG activity, and pelvic wall muscle twitch.

13. The method according to claim 1 when the method is a method for selectively modulating the EAS wherein the target pudendal nerve is an inferior rectal nerve.

14. The method according to claim 1 when the method is a method for selectively modulating the EAS wherein the target pudendal nerve innervates the external urethral sphincter.

15. The method of claim 1 wherein detecting the first physiological response and/or the second physiological response is via observation by a healthcare professional.

16. The method of claim 1 wherein detecting the first physiological response and/or the second physiological response is via feedback provided by the subject.

17. The method of claim 1 wherein the subject is under anesthesia for a duration of the method.

18. The method of claim 1 wherein the subject is conscious for a duration of the method.

19. The method of claim 1, further comprising programming a neurostimulation apparatus comprising the electrode to cause the electrode to apply the stimulatory electrical signal.

20. The method of claim 1, wherein the stimulatory electrical signal comprises a waveform having a frequency in a range of from 0.1-100 Hz.

21.-23. (canceled)

24. The method according to any preceding claim wherein the stimulatory electrical signal has an amplitude in a range of from 0.1-20 mA.

25.-34. (canceled)

35. The method according to claim 1 wherein the stimulatory electrical signal is a continuous stimulation signal.

36. The method according to claim 35 in which the stimulatory electrical signal comprises a waveform having a frequency in a range of from 1-50 Hz.

37. (canceled)

38. (canceled)
